SEQUENCE LISTING (3 sequences)

SEQ ID NO: 1 - DNA, 23 bp, Artificial Sequence
  Forward primer
ctcaactacatggtttacatgtt

SEQ ID NO: 2 - DNA, 23 bp, Artificial Sequence
  Reverse primer
gtggactccacgacgtactcagc

SEQ ID NO: 3 - DNA, 25 bp, Artificial Sequence
  Probe
cccatcaccatcttccaggagcgag

(19) United States
(12) Patent Application Publication
(10) Pub. No.: US 2015/0225790 A1
(43) Pub. Date: Aug. 13, 2015

(54) METHODS FOR IDENTIFYING SUBJECTS WITH AN INCREASED LIKELIHOOD OF RESPONDING TO CCR1 ANTAGONIST

(71) Applicant: BRISTOL-MYERS SQUIBB COMPANY, Princeton, NJ (US)

(72) Inventors: Sean Connolly, Yardley, PA (US); Susan Lynn Baglino, Hillsborough, NJ (US); Megan Wind-Rotolo, Newtown, PA (US)

(21) Appl. No.: 14/396,532

(22) PCT Filed: Apr. 18, 2013
(86) PCT No.: PCT/US2013/037079

§ 371 Date: Oct. 23, 2014

Related U.S. Application Data
(60) Provisional application No. 61/638,228, filed on Apr. 25, 2012.

(51) Int. Cl. C12Q 1/68

(52) U.S. Cl. CPC C12Q 1/6883; C12Q 2600/136; C12Q 2600/158

(57) ABSTRACT

The invention describes a method for identifying subjects with an increased likelihood of responding to treatments that modulate chemokine or chemokine receptor activity by measuring the change in expression of immune mediated genes.

### FIELD OF THE INVENTION

The invention provides novel biomarkers for identifying subjects who may have an increased likelihood of responding to CCR1 antagonist therapy.

### BACKGROUND OF THE INVENTION

Rheumatoid arthritis (RA) is characterized by the infiltration of activated inflammatory cells to the joints, contributing to hyper-proliferation and vascularization of the synovium with subsequent bone erosion. This cellular infiltrate primarily consists of activated T lymphocytes and macrophages, with a minor contribution of B cells and dendritic cells. Once localized to the disease site, these cells produce potent pro-inflammatory mediators such as chemokines, cytokines and matrix metalloproteinases that contribute to chronic inflammation and joint damage. Chemokine receptor 1 (CCR1) is expressed on these infiltrating inflammatory cells and elevated concentrations of its ligands in the synovium recruits these cells to the site of disease activity. In fact, studies show that there is a measurable increase in CCR1 positive cells in the synovium of RA patients and increased concentrations of CCL3, CCL5 and other CCR1 ligands in RA synovial fluid (Katschke et al., A&R (2001); Haringman et al., Ann. Rheum. Dis. (2006)). The magnitude of cellular infiltration in the synovium has been shown to correlate with disease severity and treatment with some anti-inflammatory therapies decreases the numbers of inflammatory cells in the joint (de Hair et al., J. Rheum. (2011); Smith, M. D. et al., Rheum. (2001)). A role for CCR1 in synovial chemotaxis was confirmed by demonstrating that ex vivo treatment of synovial fluid with a CCR1 antagonist abolishes its chemotactic activity in the majority of samples.

In addition to its role in chemotaxis, the CCR1 pathway is implicated in bone erosive pathways observed during RA. The expression of CCR1 has been measured on both osteoclasts and osteoblasts and evidence exists for expression of this receptor on chondrocytes (Hoshino et al., JBC (2010); Rauner et al., JBMR (2011)). Examination of a CCR1 knockout mouse showed altered osteoblast and osteoclast differentiation, decreased expression of transcripts related to bone metabolism, and decreased serum bone biomarkers (Hoshino et al., JBC (2010)). Furthermore, there is clinical precedence using CCR1 antagonists in the treatment of RA and other osteolytic bone diseases such as multiple myeloma (Vergunst et al., A&R (2009); Clucas et al., Clin. Pharmacokinet. (2007); Dairaghi et al. (2011), Vallet et al. (2011) Expert Opin. Ther. Targets). Although significant changes in efficacy were not observed in these studies, there were reports of changes to cellular infiltration and bone erosion (Clucas et al., Clin. Pharmacokinet. (2007)).

Current standard of care for the treatment of RA includes disease-modifying anti-rheumatic drugs that decrease immune activation and subsequent inflammation. However, a large proportion of patients are non-responsive to these medications, thus resulting in progression of disease symptoms. A CCR1 antagonist, which demonstrated potent target inhibition and an acceptable safety profile in a Phase 1 trial conducted in healthy human subjects, has progressed to a Phase 2a study in RA subjects with an inadequate response to methotrexate (MTX), where the effect of treatment on both clinical and structural responses will be evaluated. Further, identification of predictive biomarkers may allow for patient stratification in future clinical trials. Identification of patients most likely to respond favorably to the CCR1 antagonist may provide justification for use of a CCR1 antagonist earlier in the course of treatment, potentially preventing extensive structural damage of the joints.

### SUMMARY OF THE INVENTION

The invention describes a method for identifying subjects with an increased likelihood of responding to treatments that modulate chemokine or chemokine receptor activity by measuring the change in expression of immune mediated genes. The immune mediated genes are selected from the genes downstream of CCR1 signaling. The downstream genes of interest are CLEC5A (C-type lectin domain family A, member 2), MERTK (c-mer proto-oncogene receptor tyrosine kinase), SPP1 (osteopontin), TGFB1 (transforming growth factor beta 1), FCN1 (ficolin 1) and FPRL2 (formyl peptide receptor-like 2).

The invention is directed to a method for inhibiting gene expression of CLEC5A, MERTK, SPP1, TGFB1, FCN1 and FPRL2 by administering a therapeutically effective amount of a CCR1 antagonist.

The present invention is directed to identifying subjects with an increased likelihood of responding to treatment in disorders mediated by CCR1 signaling, wherein the subject has been administered a therapeutically effective amount of a CCR1 antagonist, by measuring the change in expression of immune mediated genes. The immune mediated genes are selected from the genes downstream of CCR1 signaling. The downstream genes may be selected from CLEC5A, MERTK, SPP1, TGFB1, FCN1 and FPRL2.

The invention is directed to a method of identifying antagonist of CCR1 signaling by 1) collecting whole blood at indicated time points following dosing with compound of interest, 2) stimulating the blood ex vivo, 3) determining downstream immune mediated genes levels relative to control. The immune mediated genes may be selected from CLEC5A, MERTK, SPP1, TGFB1, FCN1 and FPRL2.

### BRIEF DESCRIPTION OF THE DRAWINGS

FIGS. 1A-F show the validation of relative gene expression changes in candidate genes by follow-up qRT-PCR. Candidate genes identified by whole genome mRNA profiling were evaluated by qRT-PCR to validate gene expression changes relative to treatment. Gene expression is represented as mRNA levels relative to the untreated control sample as a function of compound concentration. Microarray results indicate that CLEC5A, MERTK, TGFB1, and FPRL2 exhibited the most significant changes in expression after 4 hours of treatment with CCR1 ligand RANTES, while SPP1 and FCN1 exhibited the most significant changes in expression after 24 hours of treatment with RANTES. Treatment of blood for follow-up qRT-PCR mirrored treatments performed for microarray. Note that FPRL2 expression was analyzed by qRT-PCR after 2 hours of treatment with RANTES (instead of 4 hours) based on assay optimization.

FIGS. 2A-D show relative CLEC5A and MERTK expression levels in a Phase I clinical trial. Healthy volunteers were treated with single or multiple doses of a CCR1 antagonist through oral administration at various dose levels; blood samples from subjects were treated ex vivo with CCR1 ligand RANTES. RNA was isolated and analyzed by qRT-PCR. Expression levels are represented as (negative) change in median CT (cycle threshold) value between the target (CLEC5A or MERTK) and the endogenous control (GAPDH) as a function of time. Results indicate that in vivo treatment with CCR1 antagonist results in the rapid and potent inhibition of CLEC5A and MERTK gene expression upregulated by ex vivo stimulation with RANTES.

FIGS. 3A-B show relative CLEC5A and MERTK expression levels in RA patient blood. Whole blood from RA patients was shipped overnight and treated with 500 nM CCR1 antagonist for 3 hours. RNA was isolated and analyzed by qRT-PCR. Expression levels are represented as mRNA levels relative to the untreated control samples as a function of treatment condition. Percent inhibition of gene expression by the investigational drug was calculated and is represented as “% change”. C-reactive protein (CRP) levels of subjects, as determined by Bioreclamation, are listed as well. Results indicate that treatment with the investigational drug decreased CLEC5A and MERTK expression levels more than 30% relative to the untreated control samples.

FIGS. 4A-B show correlation of percent inhibition of CLEC5A and MERTK expression levels by the investigational drug with CRP levels in RA patient blood. Rheumatoid Arthritis patient CRP levels were graphed as a function of the percent inhibition of gene expression levels by the investigational drug. One-tailed Pearson correlation calculations indicate that CRP significantly correlates with the percent inhibition of CLEC5A or MERTK expression by the investigational drug.

### DETAILED DESCRIPTION OF THE INVENTION

The invention describes a method for identifying subjects with an increased likelihood of responding to treatments that modulate chemokine or chemokine receptor activity by measuring a decrease in expression of immune mediated genes.

In one embodiment, the present invention is directed to identifying subjects with an increased likelihood of responding to treatments that modulate chemokine or chemokine receptor activity, wherein the subject has been administered a therapeutically effective amount of a CCR1 antagonist, by measuring a decrease in expression of immune mediated genes.

In another embodiment, the present invention is directed to identifying subjects with an increased likelihood of responding to modulation of CCR-1 receptor activity wherein the subject has been administered a therapeutically effective amount of a CCR1 antagonist by measuring a decrease in expression of immune mediated genes.

In another embodiment, the present invention is directed to identifying subjects with an increased likelihood of responding to modulation of CCR-1 receptor activity wherein the subject has been administered a therapeutically effective amount of a CCR1 antagonist by measuring a decrease in expression of immune mediated genes selected from the genes downstream of CCR1 signaling. In another embodiment, the downstream genes are selected from the group consisting of CLEC5A (C-type lectin domain family A, member 2), MERTK (c-mer proto-oncogene receptor tyrosine kinase), SPP1 (osteopontin), TGFB1 (transforming growth factor beta 1), FCN1 (ficolin 1) and FPRL2 (formyl peptide receptor-like 2).

In one embodiment, the invention provides a method for identifying subjects with an increased likelihood of responding to CCR1 antagonist, the method comprising (a) collecting whole blood from the subject, (b) stimulating the collected blood in the presence of a CCR1 antagonist, (c) measuring CLEC5A, MERTK, SPP1, TGFB1, FCN1 and/or FPRL2 gene expression, wherein said gene expression is decreased compared to non-treated reference samples.

In one embodiment, the invention provides a method for identifying subjects with an increased likelihood of responding to CCR1 antagonist, the method comprising (a) administering CCR1 antagonist to a subject, (b) collecting whole blood from the treated subject, (c) stimulating the collected blood ex vivo, (d) measuring CLEC5A, MERTK, SPP1, TGFB1, FCN1 and/or FPRL2 gene expression, wherein said gene expression is decreased compared to reference samples.

In another embodiment, the expression of immune mediated genes after treatment with CCR1 antagonist is decreased at least 2 fold when compared to reference, or decreased from 2 to 10 fold, or decreased from 2 to 8 fold, or decreased from 2 to 6 fold, or decreased from 2 to 4 fold when compared to reference.

In one aspect of the invention, the reference may be untreated blood from said subject; or a reference value determined from a representative number of nonresponsive subjects.

In another embodiment, the CCR1 antagonists are set forth and described in U.S. Pat. Nos. 7,601,844 and 8,299,098, which are incorporated by reference herein in their entirety.

In another embodiment, the present invention is directed to identifying subjects with an increased likelihood of responding to treatment in disorders mediated by CCR1 signaling, wherein the subject has been administered a therapeutically effective amount of a CCR1 antagonist, by measuring a decrease in expression of immune mediated genes, wherein said disorder is selected from osteoarthritis, aneurysm, fever, cardiovascular effects, Crohn's disease, congestive heart failure, autoimmune diseases, HIV-infection, HIV-associated dementia, psoriasis, idiopathic pulmonary fibrosis, transplant arteriosclerosis, physically- or chemically-induced brain trauma, neuropathic pain, inflammatory bowel disease, alveolitis, ulcerative colitis, systemic lupus erythematosus, nephrotoxic serum nephritis, glomerulonephritis, asthma, multiple sclerosis, arthrosclerosis, rheumatoid arthritis, restenosis, organ transplantation, psoriatic arthritis, multiple myeloma, allergies, for example, skin and mast cell degranulation in eye conjunctiva, hepatocellular carcinoma, colorectal cancer, osteoporosis, renal fibrosis, and other cancers, preferably, Crohn's disease, psoriasis, inflammatory bowel disease, ulcerative colitis, systemic lupus erythematosus, asthma, arthrosclerosis, multiple sclerosis, rheumatoid arthritis, psoriatic arthritis.

In another embodiment, the present invention is directed to identifying subjects with an increased likelihood of responding to treatment of inflammatory diseases, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of inflammatory bowel disease, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of Crohn's disease, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of psoriasis, wherein the subject has been administered a therapeutically effective amount of a CCR1 antagonist, by measuring the change a decrease of CLEC5A and MERTK genes.

In another embodiment, the present invention is directed to identifying subjects with an increased likelihood of responding to treatment of systemic lupus erythematosus, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of multiple sclerosis, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of rheumatoid arthritis, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of psoriatic arthritis, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of ulcerative colitis, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of asthma, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of atherosclerosis, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to treatment of inflammatory diseases, for example, inflammatory diseases which are at least partially mediated by CCR1, wherein the subject has been administered a therapeutically effective amount of a CCR1 antagonist, by measuring a decrease in expression of CLEC5A and MERTK genes.

In another embodiment, the present invention is directed to identifying subjects with an increased likelihood of responding to modulation of CCR1 activity wherein the subject has been administered a therapeutically effective amount of a CCR1 antagonist, by measuring a decrease in expression of CLEC5A and MERTK genes.

Identification of Immune Mediated Genes that Exhibit Decreased Expression After Treatment with CCR1 Antagonist

Gene profiling experiments to identify genes downstream of CCR1 signaling (via CCR1 ligand stimulation) were performed. It was shown that these genes were blocked following treatment with a CCR1 antagonist. Whole blood from three healthy volunteers was treated with RANTES (100 nM, CCL5), in the presence and absence of the CCR1 antagonist (50 nM, 1 μM) for 4 and 24 hours. Following the incubation, mRNA was isolated for whole genome mRNA profiling.

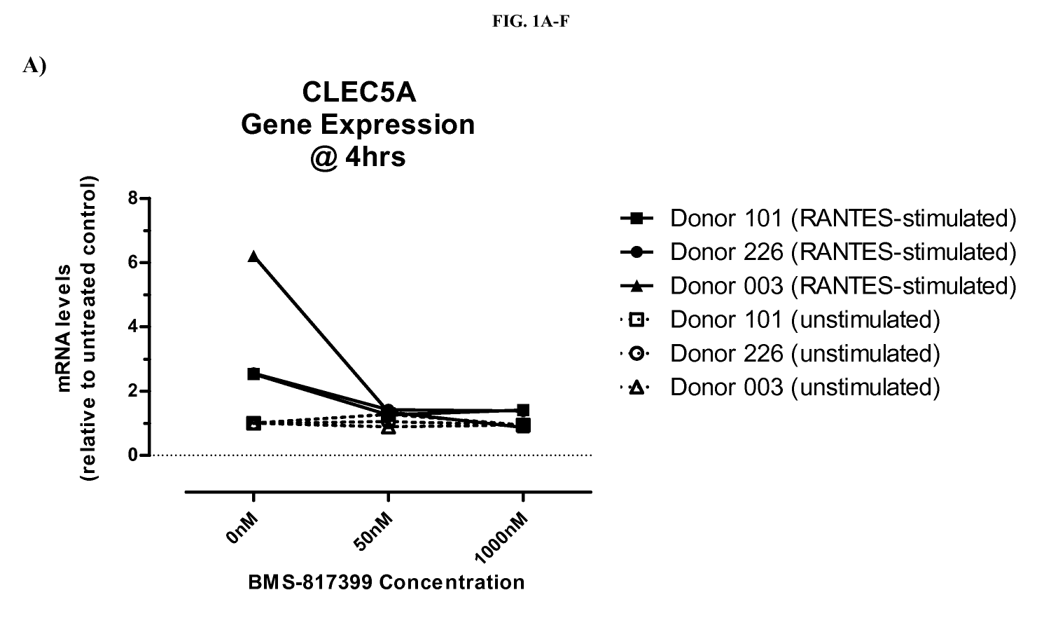

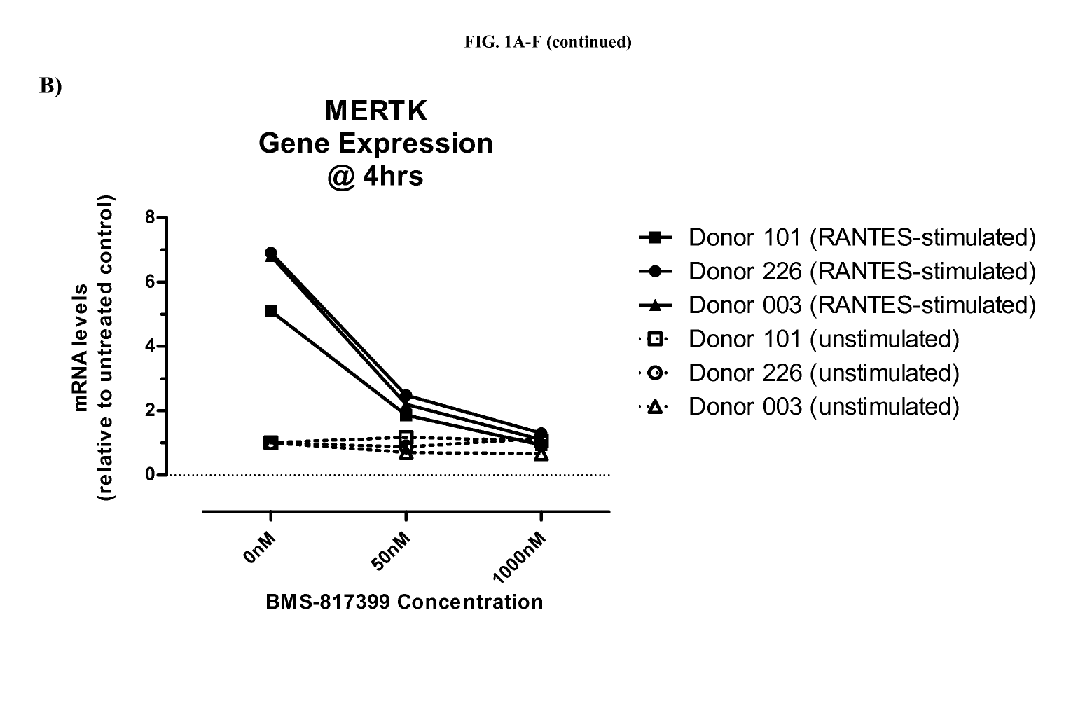

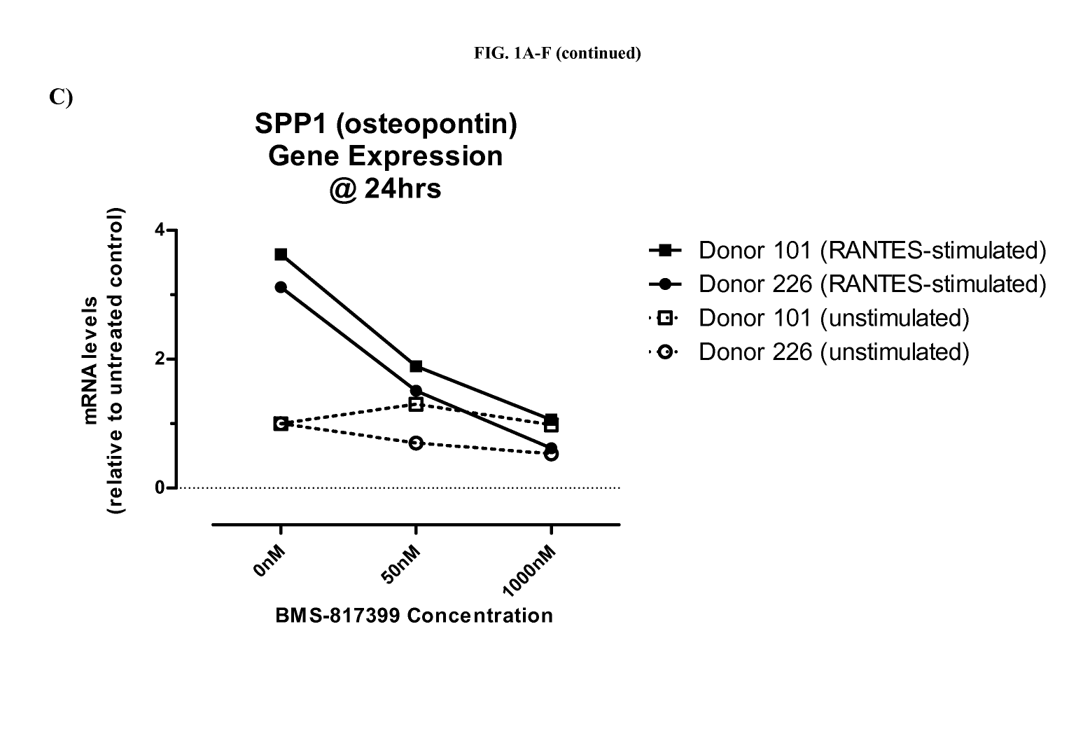

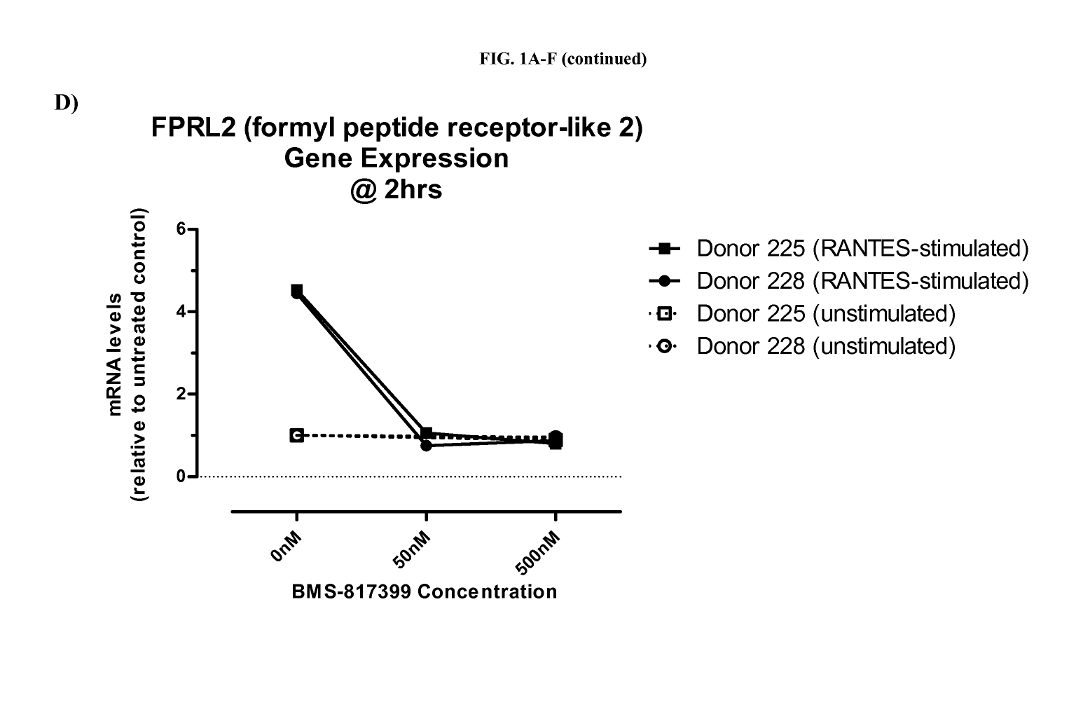

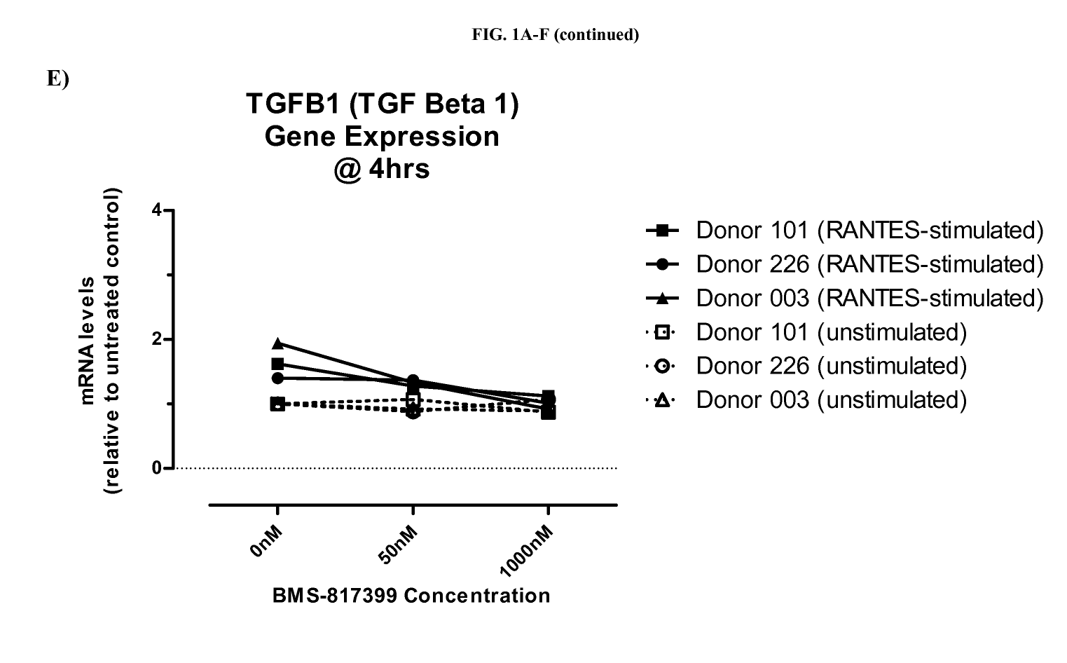

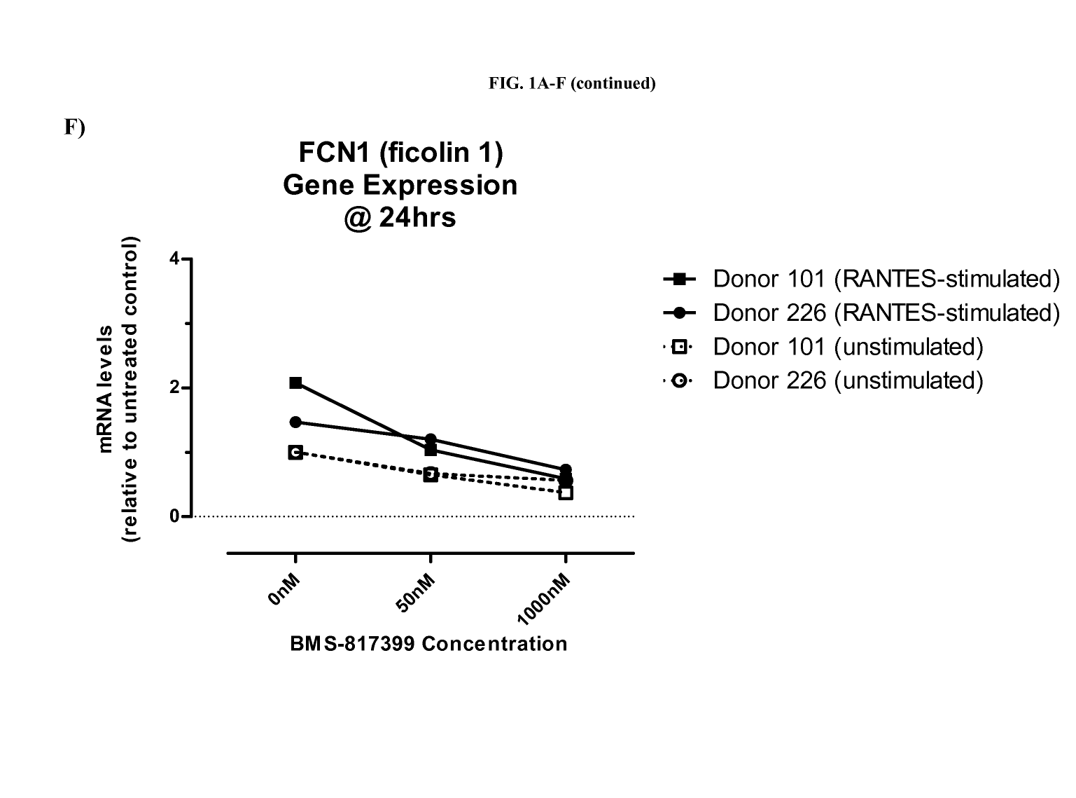

Genes that exhibited the most significant changes in expression with respect to treatment were identified (see Table 1), including the following immune-mediated genes downstream of CCR1 signaling which were blocked by the CCR1 antagonist treatment: CLEC5A (C-type lectin domain family A, member 2), MERTK (c-mer proto-oncogene receptor tyrosine kinase), SPP1 (osteopontin), TGFB1 (transforming growth factor beta 1), FCN1 (ficolin 1) and FPRL2 (formyl peptide receptor-like 2). Initial findings observed in the whole genome array experiments were validated with follow-up real-time reverse-transcription polymerase chain reaction (qRT-PCR) experiments, in which the candidate gene expression levels (normalized to that of the endogenous control GAPDH) were calculated relative to the untreated control sample for each healthy volunteer (FIGS. 1A-F). The identified genes have been implicated in autoimmunity and chronic inflammation, however their link to CCR1 has not been described.

CLEC5A is a cell surface receptor expressed on monocytes that is a key regulator of synovial injury and bone erosion during autoimmune joint inflammation (Joyce-Shaikh et al., JEM (2010)).

MERTK is a receptor tyrosine kinase that maintains immune tolerance by transducing inhibitory signals upon binding of apoptotic cells (Wallet et al., JEM (2008)). SPP1, also known as osteopontin, is an extracellular matrix protein with diverse immunomodulatory functions including bone remodeling and regulation of leukocyte migration (Haylock et al., Br. J. Haematol. (2006); Zheng et al., A&R (2009)).

This is the first time that the identified genes have been linked to CCR1 and the regulation of bone metabolism and other novel inflammatory pathways.

Phase 1 Clinical Trial where the Expression of CCR1-Related Genes was Inhibited Following Treatment of Healthy Volunteers with a CCR1 Antagonist

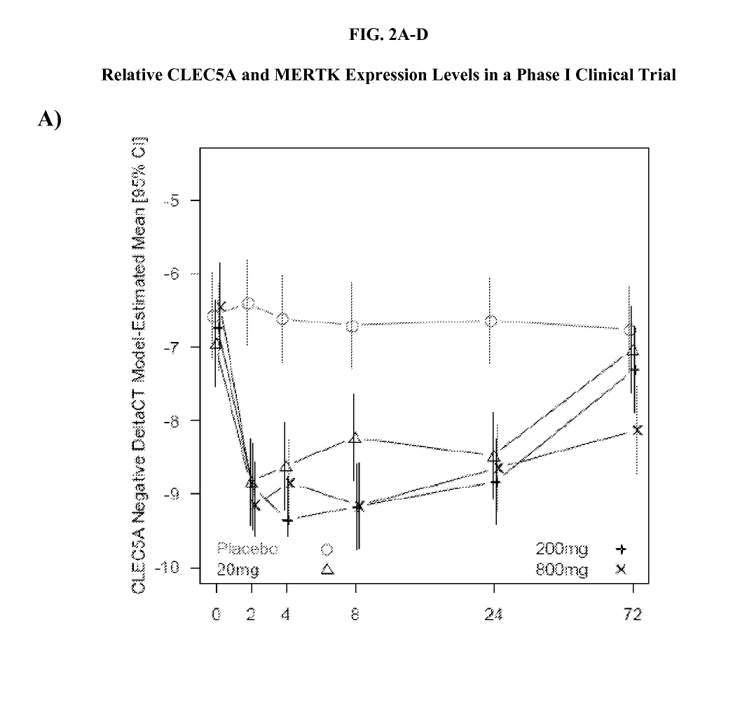

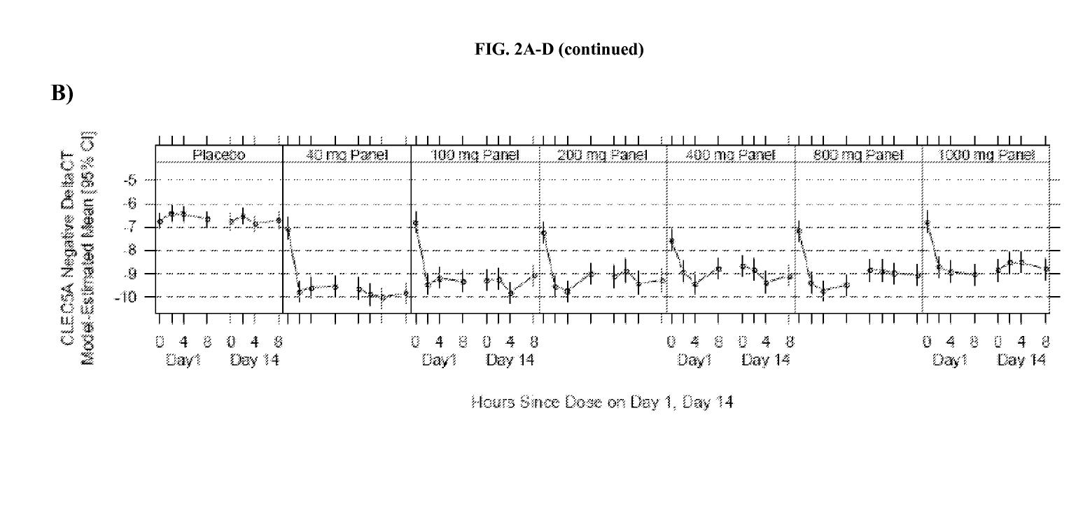

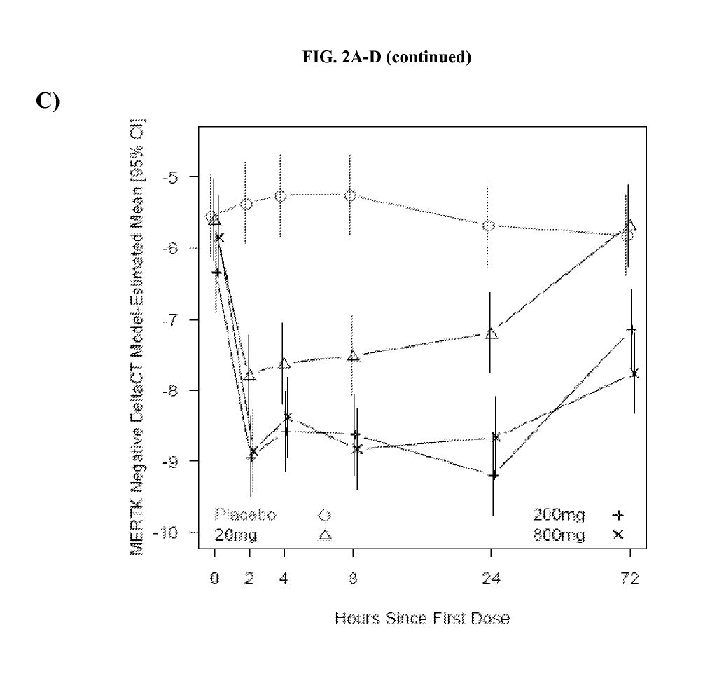

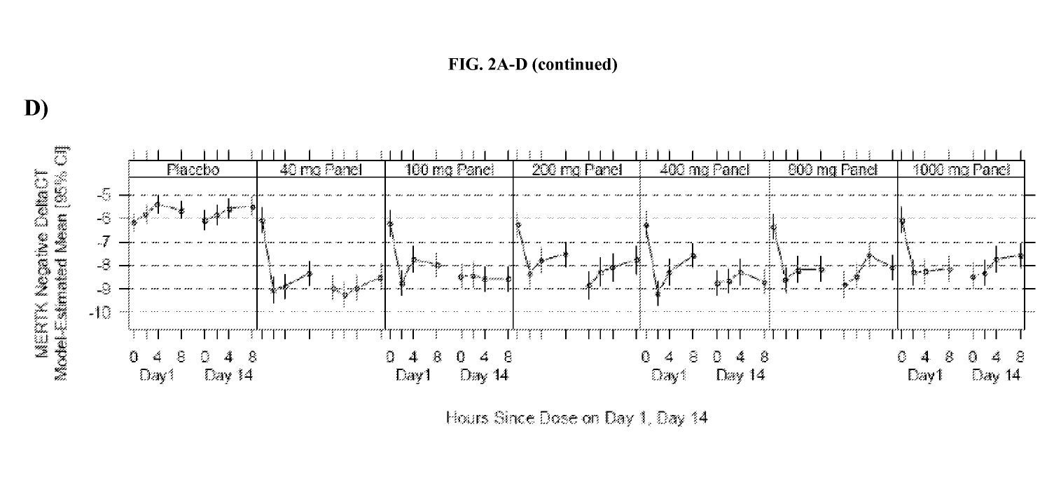

In this study (Example 2), healthy volunteers were treated with single or multiple doses of a CCR1 antagonist through oral administration at various dose levels. Pharmacodynamic effects of the compound were assessed using an ex vivo functional assay at various times post dosing. For the assay, whole blood was collected at indicated time points following dosing with the CCR1 antagonist and stimulated ex vivo for 2 hours at 37° C. with 25 nM recombinant human RANTES. After the incubation, RNA was isolated and CLEC5A and MERTK mRNA levels, relative to the GAPDH housekeeping gene, were measured by qRT-PCR in triplicate at each time-point for each subject. The median cycle time (CT) for each gene was evaluated and changes in the CT values (ΔCT) were calculated by subtracting the median GAPDH CT value from the corresponding median values for CLEC5A CT or MERTK CT. The data indicate that single and multiple dose treatments with the CCR1 antagonist resulted in the rapid and potent inhibition of CLEC5A and MERTK gene expression in the whole blood (FIGS. 2A-D). These data demonstrate in vivo neutralization of CCR1 signaling in healthy donors. Furthermore, these genes may act as informative predictive markers to identify patients most likely to respond to this therapy.

CCR1-Related Genes are Expressed in RA Whole Blood and their Expression is Regulated by the CCR1 Pathway

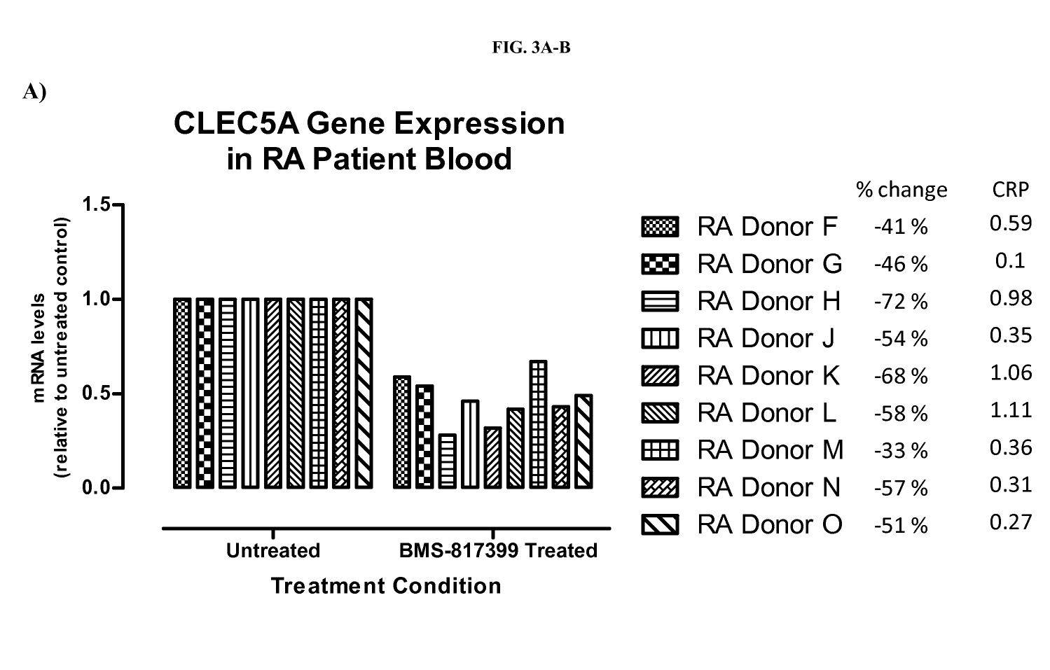

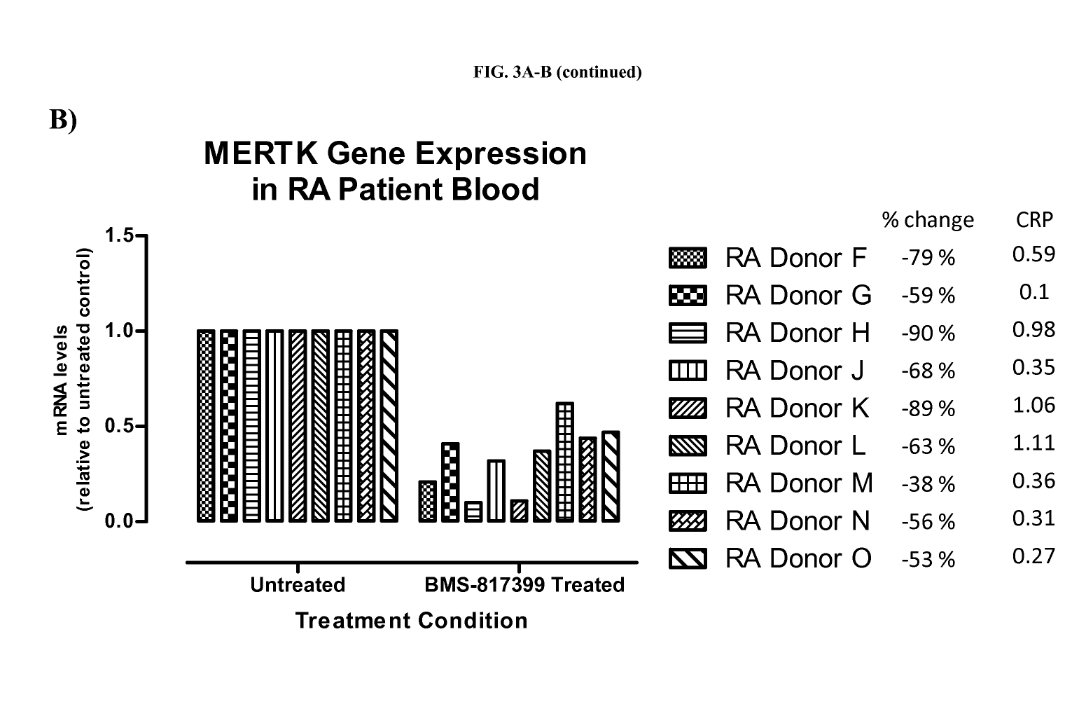

Whole blood, collected from RA patients, was stimulated with 500 nM of a CCR1 antagonist for 3 hours at 37° C. After the incubation, RNA was isolated and the gene expression levels of CLEC5A, MERTK, and the endogenous control HPRT1 were assessed by qRT-PCR for each subject. CLEC5A and MERTK mRNA levels (normalized to that of HPRT1) were calculated relative to the untreated control sample for each patient. The data demonstrate that there are measurable levels of CLEC5A and MERTK in RA patient samples and that ex vivo treatment with a CCR1 antagonist decreases the expression levels of these genes (FIGS. 3A-B).

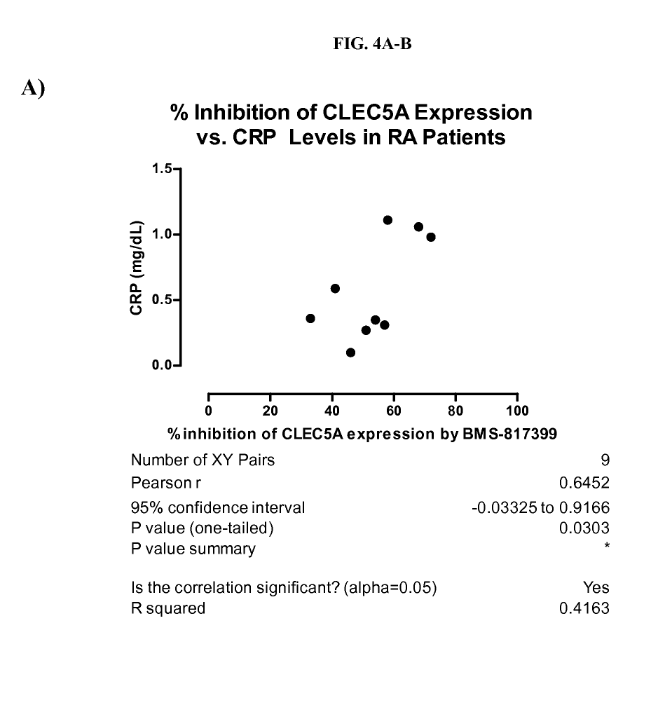

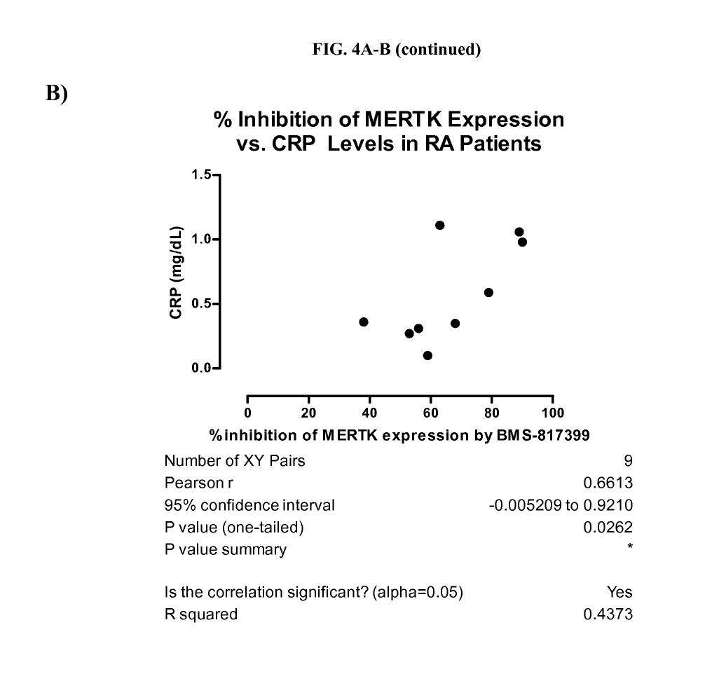

This is the first report of the expression of these genes in the whole blood of RA patients as well as the subsequent suppression of these genes by treatment with a CCR1 antagonist. Furthermore, CLEC5A and MERTK mRNA levels were associated with C-reactive protein (CRP) levels in the same patients, thus providing evidence for the correlation of these CCR1-related transcripts with a common serum-based measure of active inflammation (FIGS. 4A-B). The ongoing Phase 2a clinical trial (Example 3) will correlate the pre-treatment levels of inhibition of CLEC5A and MERTK with clinical efficacy following CCR1 antagonist exposure.

### Example 1

### Materials and Methods

Whole Blood Treatment and RNA Preparation for Whole Genome mRNA Profiling.

Lyophilized human recombinant CCR1 ligand RANTES (Peprotech, Rocky Hill, N.J.) was reconstituted to 100 μM per the manufacturer's instructions. BMS-817399 was reconstituted to 10 mM in 100% DMSO (Sigma-Aldrich, St. Louis Mo.) and further diluted 1:50 in 10% DMSO. 20× solutions of ligand and compound were prepared in Dulbecco's Phosphate-Buffered Saline (DPBS, without Mg²⁺ and Ca²⁺) (Invitrogen, Carlsbad, Calif.). For each treatment condition, a separate snap-cap polypropylene tube was prepared. Blood was first treated with the CCR1 antagonist (or DPBS), followed by treatment with ligand (or DPBS) one hour later. Treatment tubes were capped and mixed on a rotator in a 5% CO₂ incubator at 37° C. 1.5 mL treated blood was removed from each tube at 4 hours and 24 hours for RNA isolation. RNA isolation was performed per the QIAAMP® RNA Blood Mini Kit (Qiagen, Valencia, Calif.) protocol and RNA samples were concentrated per the RNeasy MINELUTE® Cleanup kit (Qiagen, Valencia, Calif.) protocol. RNA quantity and quality were assessed on the ND-8000 spectrophotometer (Thermo Scientific, Wilmington, Del.) and RNA integrity was assessed on the AGILENT® 2100 Bioanalyzer using the RNA 6000 Nano Lab Chip kit (Agilent Technologies, Santa Clara, Calif.).

Whole Genome Profiling, Statistical Analysis and Gene Selection.

RNA samples with yields ≧1.5 μg and RIN (RNA integrity number) values of 6.5 and above were randomized in 96-well plates and normalized to a concentration of 250 ng/μL in preparation for microarray analysis. Samples were shipped to the Applied Genomics group of Bristol-Myers Squibb for whole genome screening. Samples were processed on the GENECHIP® HT Array Plate Scanner with the GENECHIP® HT Human Genome U133 Array Plate Set (Affymetrix, Santa Clara, Calif.). Data obtained from the microarray analysis were analyzed by statisticians internal to Bristol-Myers Squibb. The log intensity of all valid samples was normalized to minimize nonspecific sample-to-sample variability and data were filtered to account for ligand stimulation effects, compound dose effects, and ligand-compound interactions. Box plots and volcano plots were created and utilized to best evaluate which genes exhibited the most significant changes in expression with respect to treatment (fold change>1.8, p value<0.05).

RT- and qPCR of Candidate Genes.

The resulting candidate genes identified from the microarray analysis were evaluated by qRT-PCR. Whole blood from 3 additional donors was treated and RNA was prepared as previously described. RNA was not concentrated, but was normalized to a concentration of 10 ng/μL in preparation for reverse transcription (RT) and qPCR. First-strand cDNA synthesis/RT was carried out per the QUANTITECT® Reverse Transcription kit (Qiagen, Valencia, Calif.) protocol using the GENEAMP® 9700 thermal cycler (Applied Biosystems, Foster City, Calif.). The theoretical RNA input for first strand synthesis was 5 ng/μL. qPCR was performed in a 384-well format per the QUANTITECT® Multiplex PCR kit (Qiagen, Valencia, Calif.) protocol using the ABI PRISM® 7900 real-time cycler with SDS software version 2.2 (Applied Biosystems, Foster City, Calif.). Target genes were amplified using 20×TAQMAN® Gene Expression Assays available from Applied Biosystems (see Table 2).

GAPDH served as the endogenous control for all qPCR experiments and was amplified using a custom-ordered set of primers and probe from Biosearch Technologies (see Table 3).

Samples were run in triplicate and cDNA input for qPCR was 10% of the final reaction volume. Cycling conditions were as follows: 95° C., 15 min; 45 cycles: {94° C., 60 s; 60° C., 60 s}. ROX served as the passive reference dye. Data were analyzed in the SDS software for relative quantitation. Baseline was set automatically and thresholds were set individually for each target to cross the amplification plot in the linear growth range. Amplification plots were assessed visually for each set of triplicate sample wells and outliers were omitted. Individual C_{T} values were averaged and the ΔC_{T} (Average C_{T target}−Average C_{T endogenous control}) was calculated by the software. The AΔC_{T} (ΔC_{T target}−ΔC_{T untreated control}) for each sample was calculated manually, relative to the ΔC_{T} of the untreated sample, and relative mRNA levels were calculated manually using the equation: 2^{-ΔΔCT}.

In Vitro Treatment of RA Patient Blood and Assessment by qRT-PCR.

Whole blood, drawn in EDTA VACUTAINER® tubes from RA patients, was obtained from Bioreclamation for in vitro treatment with BMS-817399 and subsequent qRT-PCR analysis. Patients were selected for blood draw if they were biologic-naive and exhibited active disease. Disease state was determined to be “active” if the patient indicated, on a scale from 1 to 10, that the arthritis pain and stiffness they felt at time of visit was 3 or higher and also that the activity level of their arthritis over the past 6 months was 3 or higher. Serum CRP and whole blood ESR levels of qualified subjects were also assessed by Bioreclamation. 25 μL 20×BMS-817399 (or 25 μL DPBS for untreated control samples) was added to 450 μL whole blood in 2 mL round-bottom tubes. Samples were mixed on a rotator in a 5% CO₂ incubator at 37° C. for 1 hour. Following compound incubation, 25 μL DPBS was added to each tube. Samples were mixed on a rotator in a 5% CO₂ incubator at 37° C. for 2 hours. The entire volume of treated blood (500 μL) was removed from each tube at 2 hours for RNA isolation. RNA was prepared as previously described. RNA was concentrated and normalized to a concentration of 20 ng/μL in preparation for reverse transcription (RT) and qPCR. First-strand cDNA synthesis/RT was carried out per the QUANTITECT® Reverse Transcription kit (Qiagen, Valencia, Calif.) protocol using the GENEAMP® 9700 thermal cycler (Applied Biosystems, Foster City, Calif.). The theoretical RNA input for first strand synthesis was 10 ng/μL. qPCR was performed in a 384-well format per the QUANTITECT® Multiplex PCR kit (Qiagen, Valencia, Calif.) protocol using the ABI PRISM® 7900 real-time cycler with SDS software version 2.2 (Applied Biosystems, Foster City, Calif.). Target genes were amplified using 20×TAQMAN® Gene Expression Assays available from Applied Biosystems (see Table 2). HPRT1 served as the endogenous control for RA patient qPCR experiments and was amplified using 20×TAQMAN® Gene Expression Assay ID Hs99999909_ml (Applied Biosystems, Foster City, Calif.). Samples were run in triplicate and cDNA input for qPCR was 10% of the final reaction volume. Cycling conditions were as follows: 95° C., 15 min; 45 cycles: {94° C., 60 s; 60° C., 60 s}. ROX served as the passive reference dye. Data were analyzed in the SDS software for relative quantitation. Baseline was set automatically and thresholds were set individually for each target to cross the amplification plot in the linear growth range. Amplification plots were assessed visually for each set of triplicate sample wells and outliers were omitted. Individual C_{T} values were averaged and the ΔC_{T} (Average C_{T target}−Average C_{T endogenous control}) was calculated by the software. The AΔC_{T} (ΔC_{T target}−ΔC_{T untreated control}) for each sample was calculated manually, relative to the ΔC_{T} of the untreated control sample, and relative mRNA levels were calculated manually using the equation: 2^{-ΔΔC}T.

### Example 2

### Relative CLEC5A and MERTK Expression Levels in Healthy Volunteers

Healthy volunteers were treated with single or multiple doses of a CCR1 antagonist through oral administration at various dose levels; blood samples from subjects were treated ex vivo with CCR1 ligand RANTES. RNA was isolated and analyzed by qRT-PCR. FIGS. 2A-D show relative CLEC5A and MERTK expression in healthy volunteers. Expression levels are represented as (negative) change in median CT (cycle threshold) value between the target (CLEC5A or MERTK) and the endogenous control (GAPDH) as a function of time. Results indicate that in vivo treatment with an CCR1 antagonist results in the rapid and potent inhibition of CLEC5A and MERTK gene expression upregulated by ex vivo stimulation with RANTES.

### Example 3

A multi-center, randomized, parallel group, double-blind, placebo-controlled, multiple oral dose proof-of-concept study in subjects with active RA having an inadequate response to treatment with MTX was performed to compare the efficacy and safety of the investigational drug to placebo on background MTX therapy over 12 weeks.

The primary study endpoint is the change from baseline in DAS28-CRP following 12 weeks of treatment and the difference in change from baseline in DAS28-CRP between the BMS-817399 and placebo groups. Secondary endpoints that assessed additional efficacy outcomes of the investigational drug over 12 weeks included ACR 20, ACR 50, ACR 70 responses and physical function (HAQ). Pharmacodynamic and biomarker assays were incorporated into the trial to inform dose selection, monitor efficacy and potentially predict treatment responses.

Subjects were randomly assigned in a 1:1:1 ratio to receive 400 mg q12h or 200 mg q12h of the investigational drug; or matching placebo for 12 weeks. After 12 weeks (84 days) of treatment, subjects were withdrawn from investigational drug product and were followed for 4 weeks to assess the safety and clinical efficacy after withdrawal of the treatment. All patients were on background therapy with MTX during the study. Subjects were seen within ±3 day time period before and after the target date for visits on Days 8, 15, 22, 29, 43, 57 and 71.

Subjects requiring washout of medication followed the recommended guidelines before completing the second screening visit. This visit occurred after the completion of the washout period as specified by the protocol and within 7 days prior to the randomization (Day 1) visit.

### Inclusion Criteria

Subjects must meet the criteria of the American Rheumatism Association (1987) or the American College of Rheumatology and European League Against Rheumatism (2010) for the diagnosis of RA.
Subjects must have an initial diagnosis of RA for at least 6 months.
Subjects must have a tender joint count of at least 6 (28 joint count), swollen joint count of at least 6 (28 joint count) and hsCRP>ULN by central laboratory value.
All subjects must have clinical evidence of synovitis in one hand/wrist at screening.
Subjects must be MTX inadequate responders: Subjects must have been treated with and tolerated MTX therapy at a weekly dose of at least 10 mg for at least 4 months prior to screening. The dose of MTX must be stable, with no change in route of administration, for at least 6 weeks prior to randomization (Day 1). Use of parenteral MTX is acceptable as clinically indicated if subjects cannot tolerate oral MTX.
Subjects must be receiving folic acid, folinic acid, or leucovorin supplementation at a stable dose for at least 4 weeks prior to Day 1 dosing
Subjects who were previously treated with up to two TNF-α inhibitors will be allowed in the study.
Subjects who consented to have an arthrocentesis must have clinically detectable synovitis (i.e., “synovial swelling”) and palpable effusion of at least one joint at baseline visit (Day 1) for collection of 1 ml of synovial fluid.
Males or females (not nursing and not pregnant) 18 years of age.

### Exclusion Criteria

Arthritis onset prior to 16 years of age.
Subjects with documented juvenile rheumatoid arthritis.
Subjects who are impaired, incapacitated, or incapable of completing study related assessments.
Subjects who are bed-or wheelchair-bound.
Subjects with other autoimmune diseases (e.g., systemic lupus erythematosus, multiple sclerosis, vasculitis) or arthritis syndromes (psoriatic arthritis, gout, Lyme disease, Reiter's syndrome).
Subjects who have any condition that could impact upon the absorption of study drug (i.e., gastric stapling, duodenal surgery, malabsorption syndrome).
Subjects with current symptoms of severe, progressive, or uncontrolled renal, hepatic, hematological, gastrointestinal, pulmonary, cardiac, neurological, or cerebral disease, or other medical conditions that, in the opinion of the investigator, might place the subject at unacceptable risk for participation in this study.
Subjects who have present or previous malignancies, except history of cured squamous or basal skin cell carcinoma or cured breast or cervical cancer for at least 5 years without evidence of recurrence.
Subjects at risk for tuberculosis (TB), specifically subjects with: a history of active TB within the last 3 years even if it was treated; a history of active TB greater than 3 years ago unless there is documentation that the prior anti-TB treatment was appropriate in duration and type; current clinical, radiographic or laboratory evidence of active TB; latent TB which was not successfully treated.
Subjects with any serious bacterial infection within the last 2 months, unless treated and resolved with antibiotics, or any clinically significant recurrent or chronic bacterial infection (such as chronic pyelonephritis, osteomyelitis and bronchiectasis).
Subjects at a high risk for systemic fungal infections (such as histoplasmosis, blastoplasmosis, or coccidioides).
Subjects with evidence (as assessed by the Investigator) of active or latent bacterial or viral infections at the time of potential enrollment, including subjects with evidence of Human Immunodeficiency Virus (HIV) infection.
Subjects who have clinically significant drug or alcohol abuse or known cirrhosis including alcoholic cirrhosis.
Subjects with severe disease likely to jeopardize the planned completion of the study (e.g., recent myocardial infarction, unstable angina pectoris, uncontrolled diabetes mellitus)
Subjects with any concurrent disease or condition that, in the opinion of the investigator, would make the subject unsuitable for participation in the study.
Subject who have received any live vaccines within 3 months of the anticipated first dose of study medication or who will have need of a live vaccine at any time during the study.
Subjects with the inability to have MRI performed; reasons include the following: magnetizable metallic parts/devices (including cardiac pacemaker) on or in the body, severe claustrophobia, body size incompatible with the scanner, or moderate to severe renal insufficiency.
   Prohibited Therapies and/or Medications
Patients who received oral or injectable azathioprine, gold, D-Penicillamine or cyclosporine in the last 30 days prior to dosing with study medication.
Patients who were treated with leflunomide in the last 6 months prior to dosing with study medication, unless an active washout with cholestyramine was performed according to the manufacturer's recommendations.
Subjects who received treatment with mycophenolate mofetil (CELLCEPT®), cyclophosphamide (CYTOXAN®), tacrolimus or other immunosuppressant in the last 3 months prior to dosing with study medication.
Subjects who received intramuscular (IM), intravenous (IV) or intra-articular (IA) glucocorticoids within 30 days prior to dosing with study medication.
Subjects who received prior treatment with more than two TNF inhibitors.
Subjects who received anakinra or etanercept in the last 30 days prior to dosing with study medication.
Subjects who received adalimumab, infliximab, golimumab, certolizumab pegol, abatacept or tocilizumab in the last 60 days prior to dosing with study medication.
Subjects who received rituximab or any B-cell depleting agent in the last 1 year prior to dosing with study medication.
Subjects who received treatment with any investigational drug in the last 30 days or less than 5 terminal half-lives of elimination, whichever is longer, prior to dosing with study medication.
Subjects who received any experimental biologic agent with an unknown half life in the last 3 months prior to Day 1 dosing.
   CLEC5A and MERTK mRNA Assessments

Transcript (mRNA) analysis was performed on whole blood collected at baseline and throughout the study to monitor the activation of CCR1 signaling pathways. In particular, two CCR1-related transcripts (CLEC5A and MERTK) was measured from mRNA isolated from whole blood using qRT-PCR methodologies. Baseline expression of these transcripts was assessed as potential predictive biomarkers.

### Serum Inflammatory and Bone-Based Biomarkers

Serum was collected for the measurement of CCR1 related ligands (CCL3, CCL5 and CCL7), and other soluble inflammatory mediators. Furthermore, markers of bone metabolism (both resorption and formation) and osteoclastogenesis was measured to explore the mechanism of action of the investigational drug in RA patients. Baseline expression of these proteins was assessed as potential predictive biomarkers.

### Synovial Fluid Assessments

Arthrocentesis of a joint with clinically detectable synovitis (i.e., “synovial swelling”) and palpable effusion on Day 1 visit sufficient for collection of 1 ml of synovial fluid was performed in subjects who consented to have this procedure. All baseline (Day 1 prior to dosing) joint assessments and MRI (only if same wrist to be imaged) were performed prior to the arthrocentesis. Synovial fluid collection was done prior to study drug administration.

Synovial fluid was collected for in vitro functional chemotaxis assays as well as the measurement of soluble CCR1 ligands. Briefly, synovial fluid is collected at baseline time point only and supernatant harvested and cryopreserved for biomarker assays to be performed at the conclusion of the study. In vitro functional chemotaxis assay were performed to measure the ability of the investigational drug to inhibit synovial fluid-induced chemotaxis of a human monocytic cell line.

Whole Blood mRNA Assessments

In addition to the PD assessments, whole blood samples were collected in PAXGENE® tubes for exploratory pharmacogenomic mRNA assessments using whole genome array techniques. These samples provide broad genomic profiling to search for novel pharmacodynamic and efficacy biomarkers related to inflammatory and/or autoimmune pathways.

TABLE 1
Whole Genome mRNA Profiling Candidate Gene List
Healthy volunteer whole blood stimulated with RANTES (100 nM) +/− BMS-817399 (1 uM)

| Probe set id | Gene Symbol | Gene Name | Mean | Fold Change | pvaluett | nlpvtt |
|---|---|---|---|---|---|---|
| 209875_s_at | SPP1 | osteopontin | −2.711 | −6.54730 | 0.03659 | 1.43664 |
| 219890_at | CLEC5A | C-type lectin 5A | −2.357 | −5.12345 | 0.00186 | 2.73033 |
| 204620_s_at | CSPG2 | versican | −2.134 | −4.39019 | 0.01342 | 1.87211 |
| 214560_at | FPRL2 | formyl peptide receptor-like 2 | −1.836 | −3.57133 | 0.05643 | 1.24852 |
| 210895_s_at | CD86 | CD86 | −1.356 | −2.55938 | 0.01436 | 1.84272 |
| 206028_s_at | MERTK | MER tyrosine kinase | −1.274 | −2.41750 | 0.00417 | 2.37946 |
| 201506_at | TGFBI | transforming growth factor beta | −1.239 | −2.36109 | 0.00349 | 2.45713 |
| 217388_s_at | KYNU | kynureninase | −1.214 | −2.32015 | 0.04633 | 1.33416 |
| 209803_s_at | PHLDA2 | pleckstrin homology-like domain A2 | −1.171 | −2.25207 | 0.04712 | 1.32683 |
| 206237_at | FCN1 | ficolin 1 | −1.16 | −2.23443 | 0.02604 | 1.58436 |
| 220832_at | TLR8 | toll like receptor 8 | −1.158 | −2.23216 | 0.01978 | 1.70387 |

TABLE 2

| Gene Symbol | Gene Name | TAQMAN ® Assay ID |
|---|---|---|
| CLEC5A | c-type lectin 5A | Hs00183780_m1 |
| MERTK | MER tyrosine kinase | Hs00179024_m1 |
| SPP1 | osteopontin | Hs00959010_m1 |
| FPRL2 | formyl peptide receptor-like 2 | Hs00266666_s1 |
| TGFB1 | transforming growth factor | Hs00932734_m1 |
| | beta | |
| FCN1 | ficolin 1 | Hs00157572_m1 |

TABLE 3
GAPDH primer-probe set.

| | | | | Working |
|---|---|---|---|---|
| | | 5′ | 3′ | Concen- |
| | | modifi- | modifi- | tration |
| | Sequence | cation | cation | (20×) |
| Forward | 5′-CTCAACTACATGG | N/A | N/A | 1.6 μM |
| | TTTACATGTT-3′ | | | |
| Reverse | 5′-GTGGACTCCACGA | N/A | N/A | 1.2 μM |
| | CGTACTCAGC-3′ | | | |
| Probe | 5′-CCCATCACCATCT | CAL Fluor | BHQ-1 | 3 μM |
| | TCCAGGAGCGAG-3′ | Orange 560 | | |

1. A method for identifying subjects with increased likelihood of responding to CCR1 antagonists comprising the steps of (a) obtaining a sample of blood from a patient (b) stimulating the cells ex viva in the presence of a CCR1 antagonist (c) analyzing gene expression levels of C-type lectin domain family A, member 2 (CLEC5A), c-mer proto-oncogene receptor tyrosine kinase (MERTK), osteopontin (SPP1), transforming growth factor beta 1 (TGFB1), ficolin 1 (FCN1) and/or formyl peptide receptor-like 2 (FPRL2), (e) comparing expression levels from treated samples against untreated control samples wherein samples with decreased expression levels indicate an increased likelihood of responding to the CCR1 antagonist

2. The method according to claim 1 wherein CLEC5A gene expression level is analyzed.

3. The method according to claim 1 wherein MERTK gene expression level is analyzed.

4. The method according to claim 1 wherein SPP1 gene expression level is analyzed.

5. The method according to claim 1 wherein TGFB gene expression level is analyzed.

6. The method according to claim 1 wherein FCN1 gene expression level is analyzed.

7. The method according to claim 1 wherein FPRL2 gene expression level is analyzed.

8. The method according to claims 1-7 wherein the gene expression level is decreased at least 2 fold, from 2 to 10 fold, from 2 to 8 fold, from 2 to 6 fold or from 2 to 4 fold when compared to the reference.

9. A method of inhibiting the gene expression of CLEC5A, MERTK, SPP1, TGFB, FCN1 and/or FPRL2 in a subject with immune disorders mediated by CCR1 signaling wherein said method comprises the administration of a therapeutically effective amount of a CCR1 antagonist.

10. A method for identifying subjects with increased likelihood of responding to CCR1 antagonists comprising the steps of (a) administering a therapeutically effective amount of a CCR1 antagonist to said subject (b) obtaining a sample of blood from the treated subject (c) stimulating the cells ex vivo (d) analyzing gene expression levels of CLEC5A, MERTK, SPP1, TGFB, FCN1 and/or FPRL2 (e) comparing expression levels from treated samples against untreated control samples wherein samples with decreased expression levels indicate an increased likelihood of responding to the CCR1 antagonist.

11. The method according to claim 10 wherein CLEC5A gene expression level is analyzed.

12. The method according to claim 10 wherein MERTK gene expression level is analyzed.

13. The method according to claim 10 wherein SPP1 gene expression level is analyzed.

14. The method according to claim 10 wherein TGFB gene expression level is analyzed.

15. The method according to claim 10 wherein FCN1 gene expression level is analyzed.

16. The method according to claim 10 wherein FPRL2 gene expression level is analyzed.

17. The method according to claims 10-16 wherein the gene expression level is decreased at least 2 fold, from 2 to 10 fold, from 2 to 8 fold, from 2 to 6 fold or from 2 to 4 fold when compared to the reference.

18. A method for identifying subjects with an increased likelihood of responding to treatment in immune disorders mediated by CCR1 signaling, wherein the subject has been administered a therapeutically effective amount of a CCR1 antagonist, comprising measuring a decrease in expression of immune mediated genes where the immune disease is selected from the group consisting of osteoarthritis, aneurysm, fever, cardiovascular effects, Crohn's disease, congestive heart failure, autoimmune diseases, HIV-infection, HIV-associated dementia, psoriasis, idiopathic pulmonary fibrosis, transplant arteriosclerosis, physically- or chemically-induced brain trauma, neuropathic pain, inflammatory bowel disease, alveolitis, ulcerative colitis, systemic lupus erythematosus, nephrotoxic serum nephritis, glomerulonephritis, asthma, multiple sclerosis, arthrosclerosis, rheumatoid arthritis, restenosis, organ transplantation, psoriatic arthritis, multiple myeloma, allergies, for example, skin and mast cell degranulation in eye conjunctiva, hepatocellular carcinoma, colorectal cancer, osteoporosis, renal fibrosis, and other cancers.
